(19) United States
(12) Patent Application Publication
(10) Pub. No.: US 2014/0207116 A1
(43) Pub. Date: Jul. 24, 2014

(54) Implantable High Flow Dual Lumen Multi-Window Vascular Access Port Catheter

(71) Applicant: Grit Armstrong Adler, Tampa, FL (US)

(72) Inventor: Grit Armstrong Adler, Tampa, FL (US)

(21) Appl. No.: 14/156,948

(22) Filed: Jan. 16, 2014

(51) Int. Cl. A61M 1/36; A61M 39/10

(52) U.S. Cl. CPC A61M 1/3653; A61M 39/10
USPC 604/533; 604/288.02

(57) ABSTRACT

This improved subcutaneous implantable vascular access port invention is designed to accommodate a larger catheter caliber attachment and six multi-window elongated dual lumen trough reservoir to allow 200-600 milliliters per minute of blood flow. This accommodating high flow fluid volume blood flow rate will be ideal to support functions to include but not limited to dialysis and electrophoresis. Both the six multi-window non-coring needle access septum and subcutaneous implantation of this high flow vascular access port will markedly reduce the risk of infection. The augmented port length trough style and six self-sealing multi septum windows will allow variable needle placement on a bi-daily basis. The purpose of the variable different location needle site is to avoid both skin breakdown, and reduce the risk of infection.

This invention is a subcutaneous implantable dual lumen high flow volume vascular port catheter, designed to rapidly both deliver fluid into and withdraw fluid from the patients bloodstream. Access into the solid based reservoir dual lumens is via a non-coring needle through an anterior non-coring needle penetrable septum. This septum is self-sealing, allowing for multiple repeated non-coring needle placement without leaking. The septum holds and seals the non-coring needle in place during fluid movement. The septum is designed for subcutaneous tactile palpation for localization of non-coring needle placement. The septum has a trough rectangular shape with six windows in which to access the lumens.

Within the base of each of the two lumens are large calibur outlet stems that connect a high pressure catheter from the reservoir lumen through the catheter into the patients bloodstream. Flushing positive pressure through the needle into the reservoir would allow fluids to be sent into the patients blood stream. Visa versa aspiration negative pressure through the needle within the reservoir will allow blood to be removed from the blood stream.

The caliber of the outlet stems and high pressure catheter caliber is specifically designed to allow large volume high flow rates to accommodate for functions such as but not limited to dialysis.

### BRIEF DESCRIPTION OF THE DRAWINGS

The associated drawings illustrate the invention:

FIG. 1 is an exterior oblique perspective view of the implantable high volume flow rate dual lumen vascular access port (10) of my new design. (11) is the needle impenetrable base housing. (21,22) are the septum windows for non-coring needles to access the lumens.

FIG. 2 is an exterior anterior top view of (10) illustrated in FIG. 1. Once implanted subcutaneously, this anterior side will abut the subcutaneous skin for tactile palpation of the septum windows (21,22).

FIG. 3 is a lateral side view of (10) illustrated in FIG. 1

FIG. 4 is a non-catheter side view of (10) illustrated in FIG. 1

FIG. 5 is a non-catheter side view of (10) illustrated in FIG. 1

FIG. 6 is a posterior back base view of (10) illustrated in FIG. 1

FIG. 7 is a cut side view of (10) illustrated in FIG. 2. Demonstrating the reservoir lumen (24) in open direct communication with the catheter. The supporting solid struts (25) supporting the overlying septum (21,22).

FIG. 8 is a cut anterior view of (10) illustrated in FIG. 3. Demonstrating that the supportive struts (25) are contiguous with the solid base (11). Both lumens (23,24) are in open direct communication with the catheter.

FIG. 9 is a axial cut view of (10) illustrated in FIG. 1 catheter.

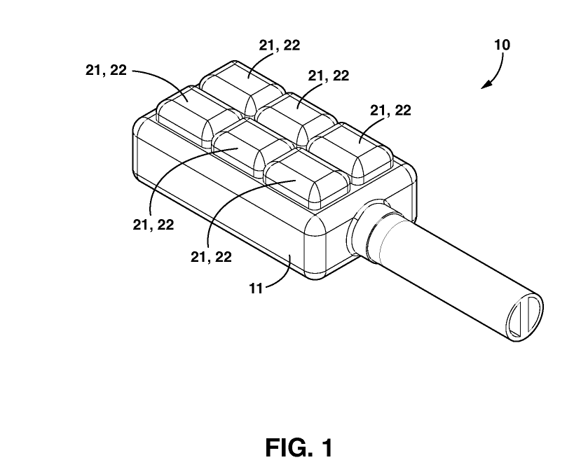

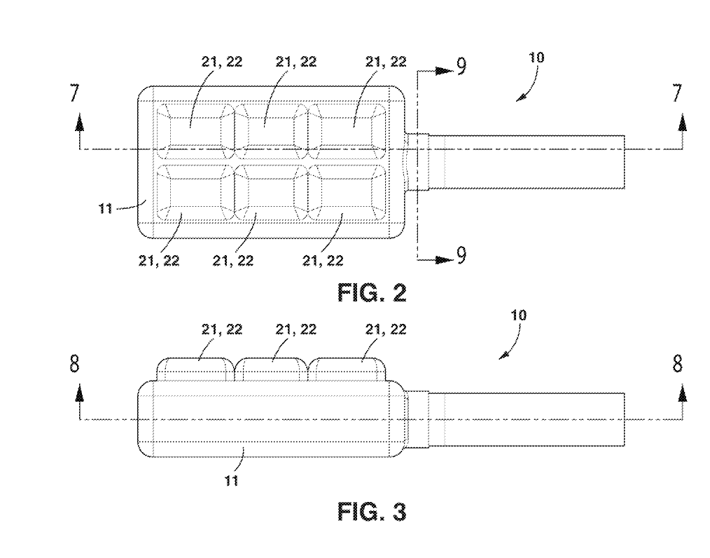

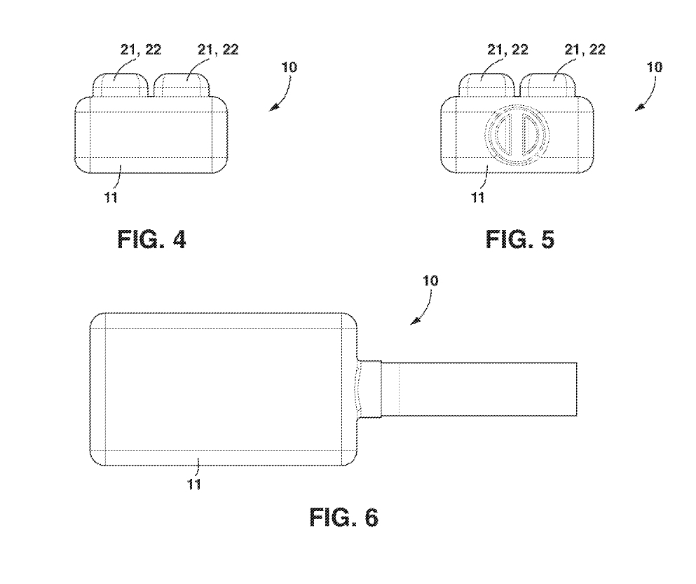

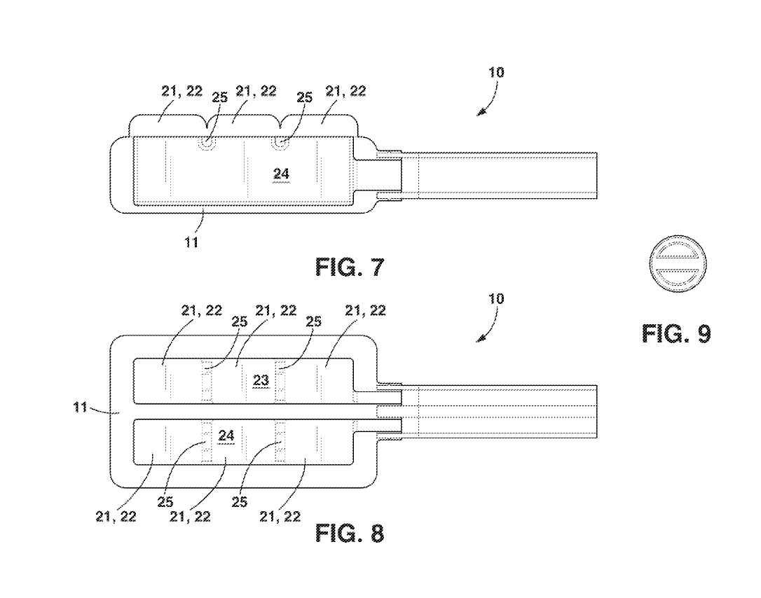

### SUMMERY OF THE INVENTION

This invention improves implantable dual lumen vascular access port catheters by creating a high volume flow rate, high pressure system with larger caliber reservoir stems and large caliber catheter with six windows septum for needle access. Aside from the standard subcutaneous vascular access port benefits, this design will allow for dialysis vascular access to be performed. Which vascular dialysis demands high flow rate volumes.

The vascular dialysis patients typically receive dialysis access every other day. The repeated subcutaneous needle sticks every other day in the same skin location overlying the standard two window vascular access ports; tend to cause skin breakdown, thinning and high risk of infection. Hence, once the skin is thinned and infection happens, the standard port would need to be removed. This six window port invention would allow for the standard every other day needle access point to be distributed along the skin in a different window location evenly between the six windows; instead of the standard port two skin locations. Hence, allowing for the proper subsequent needle stick adjacent window selection, the same window in this invention would not be needle accessed but once a week; instead of the standard every other day. This would allow sufficient time for the overlying subcutaneous skin to heal fully before the next needle access one week later.

Ideally the purpose of this invention is to allow the patient the option of blood dialysis without the need of surgery to create standard vascular fistulas or graft fistulas. Also, allows for patients who are not and/or no longer surgical candidates to receive blood dialysis via this port catheter invention.

1. Dual lumen elongated trough style reservoir with six multi-window septum subcutaneous implantable vascular access port with larger caliber stems.

2. The access port of claim 1, a solid base made of material impenetrable to a needle.

3. The access port of claim 2, the base has a central wall that separates and creates the two trough lumens.

4. The access port of claim 3, each lumen wall anteriorly has two perpendicular supporting thin struts that lay on top of the walls.

5. The access port of claim 4, The two struts per lumen trifurcate the top of each lumen and along with the top of the base walls they support the self-sealing septum. The septum is placed on top of the walls and struts. This configuration creates a total of six windows for non-coring needle access. Three access windows per lumen.

6. The access port of claim 5, the septum anteriorly is tri-lobed in a trough formation overlying each lumen. This allows for tactile palpation once under the skin to select one of the six windows for non-coring needle access.

7. The access port of claim 5, the septum is locked into place with a cap that is shaped to the orientation of the base creating a fluid sealed six window dual lumen port.

8. The access port of claim 3, each lumen has an outlet stem caliber that is large enough to allow for flow volumes high enough for procedures such as dialysis; as well as for low flow volumes such as medication/fluids.

9. The access port of claim 8, The outlet systems can come preassembled connected to a high pressure catheter locked onto the outlet stems.

10. The access port of claim 9, the catheter can come with a predetermined length.

11. The access port of claim 9, the high pressure catheter can come separately and be attached to the port outlets after the necessary catheter length has been determined and cut to accommodate patient anatomy and body location.

12. The access port of claim 11, a sliding plastic lock that slides along the catheter will lock the catheter into place into the port. Thus creating a high pressure seal between the catheter and port lumens.
